(19) United States
(12) Patent Application Publication
(10) Pub. No.: US 2017/0156671 A1
(43) Pub. Date: Jun. 8, 2017

(54) BIOWEARABLE EMBEDDED CIRCUIT

(71) Applicant: Accenture Global Solutions Limited, Dublin (IE)

(72) Inventors: Eric Schneider, Austin, TX (US); Matthew A. Murray, Austin, TX (US); Eric Bee, Austin, TX (US); Philippe Moore, Portland, OR (US); Marc Boudria, Austin, TX (US); Ben E. Lamm, Dallas, TX (US); Andrew Busey, Austin, TX (US)

(21) Appl. No.: 15/365,357

(22) Filed: Nov. 30, 2016

Related U.S. Application Data
(60) Provisional application No. 62/264,663, filed on Dec. 8, 2015.

(51) Int. Cl. A61B 5/00; A61B 5/145; A61B 5/024; A61B 5/0488; A61B 5/11; A61B 5/01

(52) U.S. Cl. CPC A61B 5/6832; A61B 5/1123; A61B 5/01; A61B 5/02438; A61B 5/0488; A61B 5/4875; A61B 5/4266; A61B 5/14546; A61B 5/0015; A61B 2560/0214; A61B 2560/0242

(57) ABSTRACT

Methods, systems, and apparatus, including computer programs encoded on a computer storage medium, for establishing a communication connection between a biowearable embedded circuit and a computing device associated with a user, wherein the biowearable embedded circuit is mounted to the user; obtaining atmospheric, motion, communication, and/or biometric data associated with the user; and providing the atmospheric, motion, communication, and/or biometric data to the computing device.

### CROSS-REFERENCE TO RELATED APPLICATION

This application claims priority to U.S. Provisional Patent Application No. 62/264,663 filed on Dec. 8, 2015, entitled “Biowearable Embedded Circuit,” which is hereby incorporated by reference in its entirety.

### BACKGROUND

This disclosure relates to biowearable electronics, specifically, skin-mounted atmospheric, motion, communication, and/or biometric data-collection and transmission wearable electronics, that may be of a temporary nature.

### SUMMARY

Innovative aspects of the subject matter described in this specification may be embodied in methods that include actions establishing a communication connection between a biowearable embedded circuit and a computing device associated with a user, wherein the biowearable embedded circuit is mounted to the user; obtaining atmospheric, motion, communication, and/or biometric data associated with the user; and providing the data to the computing device.

Another aspect includes the biowearable embedded circuit including a first conductive layer that transmits electrical signals between a sensor module and one or more components of the biowearable embedded circuit; a second adhesive component layer adhering the biowearable embedded circuit to a skin of a user; and a third insulation layer to insulate the biowearable embedded circuit, wherein the sensor module is coupled to the first conductive layer to obtain atmospheric, motion, communication, and/or biometric data associated with the user. Another aspect includes the second adhesive component layer comprising a water-soluble adhesive.

Another aspect includes a method of manufacturing the biowearable embedded circuit, including generating a graphics file of the biowearable embedded circuit, the graphics file including one or more layers of the biowearable embedded circuit; printing the graphics file to produce each layer of the biowearable embedded circuit in a specified order; and attaching one or more components to the printed biowearable embedded circuit.

Other implementations of these aspects include corresponding systems, apparatus, and computer programs, configured to perform the actions of the methods, encoded on computer storage devices.

Implementations of the present disclosure provide one or more of the following example advantages. The biowearable embedded circuit provides a reduction in form factor of wearable electronics to provide seamless monitoring of user biometric data. The biowearable embedded circuit provides direct interface with a mobile communications device (e.g., smartphone) to provide non-intrusive monitoring of the user's data.

The details of one or more implementations of the subject matter described in this specification are set forth in the accompanying drawings and the description below. Other potential features, aspects, and advantages of the subject matter will become apparent from the description, the drawings, and the claims.

### BRIEF DESCRIPTION OF THE DRAWINGS

FIG. 1 depicts an example system that can execute implementations of the present disclosure.

FIG. 2 depicts an example process for production of a biowearable embedded circuit.

FIG. 3 depicts an example use of a biowearable embedded circuit.

FIG. 4 is side view of a biowearable embedded circuit.

Like reference numbers represent corresponding parts throughout.

### DETAILED DESCRIPTION

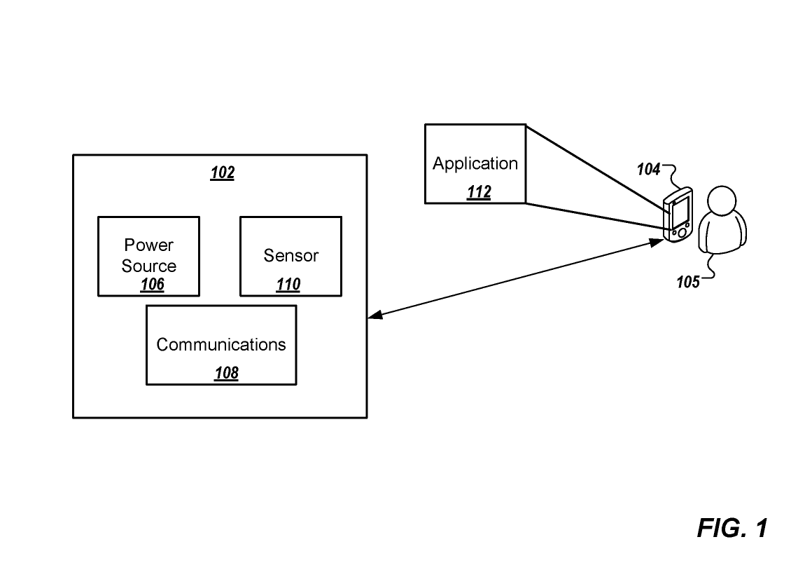

FIG. 1 depicts an example system 100 that can execute implementations of the present disclosure. In general, the system 100 includes a biowearable embedded circuit (BEC) 102 and a computing device 104. The BEC 102 may be intended to be of a temporary nature, that is, akin to a temporary tattoo. The BEC 102 includes a power source component 106, a communications module 108, and a sensor module 110. In some implementations, the BEC 102 includes other modules (not shown) such as a processor, memory, and a bus. In some examples, the BEC 102 and the computing device 104 are associated with a user 105. That is, the BEC 102 can be coupled with the user 105 (e.g., the BEC 102 is mounted to the user 105 utilizing the skin of the user 105) and the user 105 utilizes the computing device 104. In some examples, the BEC 102 is flexible. In some examples, the BEC 102 is of a size of approximately 10 millimeters by 10 millimeters (or smaller).

In some implementations, the power source component 106 provides power (e.g., electrical energy) to the BEC 102 and the components thereof (e.g., the communications module 108 and the sensor module 110). In some examples, the power source can provide power directly (e.g., via electrochemical cells) or indirectly (e.g., provide access to power). In some examples, the power source component 106 provides power by obtaining (harvesting) energy from the user 105. That is, the power source component 106 can obtain energy that is produced (actively or passively) by the user 105. In some examples, the power source component 106 obtains energy from the user 105 by one or more of muscle energy (e.g., movement of the muscles of the user 105—the muscle associated with the portion of the skin the BEC 102 is mounted to), kinetic energy (e.g., walking movement of the user 105), heat energy (e.g., heat produced by the user 105), and flex energy (e.g., contraction of the muscles of the user 105—thus muscle associated with the portion of the skin the BEC is mounted to).

In some examples, the power source competent 106 includes a coin/button cell battery, a lithium polymer (LiPo) battery, or any other type of battery. In some examples, the power source component 106 includes a low power (e.g., 3.3-volt) battery. In some examples, the power source component 106 includes (or is associated with) a step-up generator (e.g., 5-volt—for utilization by power peripheral devices associated with the BEC 102).

In some implementations, the BEC 102 communicates with the computing device 104 using the communications module 108. The communications protocol utilized by the communications module 108 can include any communications protocol, such as Bluetooth, Bluetooth low energy, wireless (e.g., WLAN or Wi-Fi), or radio frequency. The communications module 108 can provide obtained sensor data to the computing device 104, described further below.

In some implementations, the sensor module 110 obtains data associated with the user 105. That is, the sensor module 110 obtains data associated with the user 105 through contact with the skin of the user 105, e.g., data that can be obtained at the surface of the skin of the user 105. In some examples, the sensor module 110 (and/or a processor associated with the sensor module 110) can analyze the obtained data to determine calculated data. That is, the sensor module 110 (and/or a processor associated with the sensor module 110) process the obtained data, and apply one or more algorithms to the data to obtained further refined data. In some examples, the sensor module 110 is a microcontroller.

In some examples, the data that is obtained by the sensor module 110 that is associated with the user 105 can include acceleration (e.g., walking, driving), flex-based data (e.g., bend, stretch, twist), capacitive touch-based data, temperature, pulse, heart rate, EMG, hydration, sweat, blood-alcohol content level, or any biometric based-data of the user.

In some examples, the sensor module 110 can obtain data associated with an environment of the user 105. That is, the sensor module 110 obtains data associated with the user 105 through the environment of the user 105, e.g., data that can be obtained of the environment that is proximate to the user 105. In some examples, the data that is obtained by the sensor module 110 that is associated with an environment of the user 105 can include barometric pressure, gyroscope based data, force based data, temperature, sound hall effect based data, radiation, moisture, infrared beam based data, tactile based data, proximity based data, humidity, viscous fluid pressure, ultraviolet light based data, RGB light based data, and chemical/gas data (e.g., carbon monoxide, methane gas, LPG Gas, hydrogen gas), and electric resistance.

In some implementations, the computing device 104 is a mobile computing device, e.g., a smartphone, a tablet computing device, a smart watch, a wearable computing device, a touchscreen laptop, a touchscreen desktop. In some examples, the computing device 104 includes a graphical user interface (GUI). A number of components within the computing device 104 provide for interaction with the computing device 104. In some examples, an application 112 resides on the computing device 104. That is, the computing device 104 provides/enables execution of the application 112.

To that end, the computing device 104 can receive data from the BEC 102 (e.g., via the communications module 108) and appropriately processes the data. For example, the application 112 can appropriately process the data from the BEC 102 and provide for display via the GUI the data in a user-friendly format that the user 105 can interact with. For example, the application 112 can provide for display raw data to the user 105, and/or provide the data represented graphically. The application 112 can also calculate refined data from the data received from the BEC 102, and provide for display this data via the GUI. In some examples, the application 112 can monitor the received data from the BEC 102 and compare such to thresholds (e.g., auto-generated or user-generated). Based on the comparison, the application 112 can provide a notification via the GUI to the user 105 (e.g., push notification) of an alert (e.g., a temperature data point obtained from the BEC 102 is above a threshold).

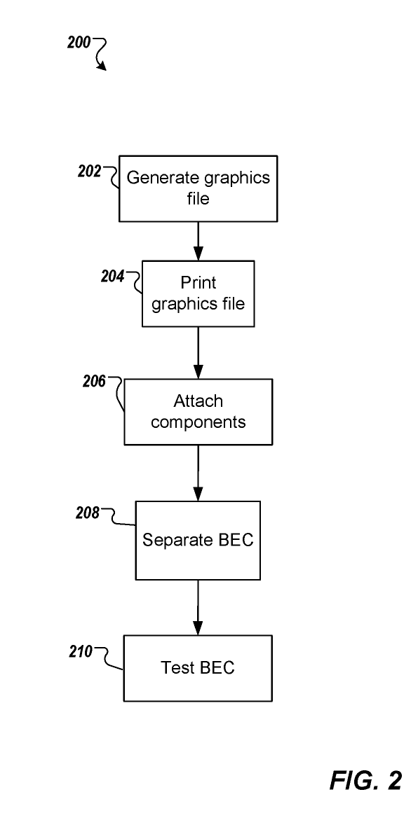

FIG. 2 is a flowchart of an example process 200 for production of the BEC 102. The example process 200 can be executed using one or more computing devices. A graphics file is generated for the BEC (202). In some examples, the graphics file is associated with a file format such as Portable Networks Graphics (PNG) or Graphics Interchange Format (GIF). In some examples, the graphics file includes three layers: a conductive layer, an adhesive component layer, and an aesthetic layer. In some examples, the adhesive component layer is positioned between the conductive layer and the aesthetic layer. In some examples, the aesthetic layer is polyethylene terephthalate (PET) based (e.g., “Mylar”). The PET of the aesthetic layer provides electric insulation, e.g., from the environment of the user 105. In some examples, the aesthetic layer includes a specific image.

In some examples, the graphics file includes an additional layer positioned such that when the BEC 102 is applied to the skin of the user 105, the additional layer is positioned between the conductive layer and the skin of the user 105. The additional layer may minimize, if not prevent, amperage loss of the BEC 102 (e.g., as a result of skin resistance).

The BEC as the graphics file is printed (204). Specifically, a printer (e.g., a modified ink jet printer) prints the graphics file such that each layer of the graphics file is printed using the specified materials. In some examples, the conductive layer is printed on a barrier (base layer), the adhesive component layer is printed on the conductive layer, and the aesthetic layer is printed on the conductive layer. In some examples, the adhesive component layer comprises a water-soluble adhesive. In some examples, the composition of the adhesive component layer is selected so as to allow for the temporary application of the BEC 102 to the skin of the user 105, e.g., the adhesive component layer is dissolvable, or removable. In some examples, the barrier (base layer) includes a water-slide paper or adhesive paper.

Components are attached to the printed BEC (206). Specifically, the printed BEC is positioned within a plotter device such that the components are placed onto the circuit at appropriate positions. In some examples, the components include a communications module, a power source component, and a sensor module. In some examples, the sensor module is based on the data to be obtained from the user 105 and/or the environment of the user 105.

The BEC is separated (208). Specifically, the BEC is placed in a laser cutter device such that when the printed sheet containing the BEC includes multiple BECs, each individual BEC is separated (e.g., removed) from the remaining BECs.

The BEC is tested (210). Specifically, the BEC is tested against criteria, including testing for electrical faults.

In some examples, the conductive layer includes copper material to i) transmit electrical signals between the components of the BEC 102 and ii) connect the components of the BEC 102. In some examples, the conductive layer includes an electrically conductive material (e.g., gel). For example, the electrically conductive material of the conductive layer can be permeable/flexible in an initial state (e.g., during initial formation of the BEC 102—printing of the graphics file). As such, when the components are attached to the BEC 102, connections between the components and the electrically conductive material is facilitated. Furthermore, as time lapses, the electrically conductive material may transition to a different state such that the conductive layer is in a non-permeable/hardened state. As such, the aforementioned connections are solidified, while the BEC 102 remaining flexible.

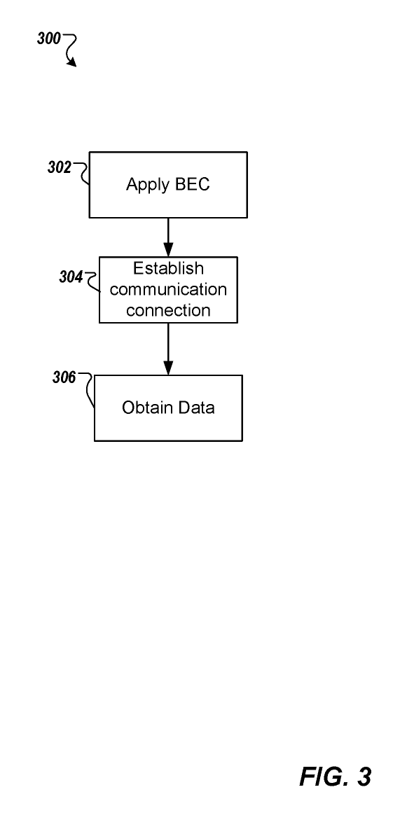

FIG. 3 is a flowchart of an example process 300 for use of the BEC 102. The example process 300 can be executed using one or more computing devices. The BEC 102 is applied (mounted) to the user (302). Specifically, the BEC 102 is applied to be in contact with the skin of the user 105. In some examples, the positioning of the BEC 102 is based on the type of data to be collected from the user 105 and/or the environment of the user 105. That is, different portions of the skin of the user 105 may provide a more optimal placement of the BEC 102 based on the desired data to be obtained.

In some examples, to apply (mount) the BEC 102 to the user 105, the user 105 removes a backing (e.g., protective layer) of the BEC 102 and positions the BEC 102 adjacent the skin of the user 105. The BEC 102 is adhered (mounted) to the skin of the user 105 by soaking the BEC 102 with water to transfer the BEC 102 to the skin of the user 102 such that the BEC 102 is adhered to the skin of the user 105. In some examples, the BEC 102 includes a removable (e.g., sticker) form factor. Thus, the user 105 is able to remove the backing (e.g., protective layer) of the BEC 102 and position the BEC 102 adjacent the skin of the user 105 to adhere (mount) the BEC 102 through adhesion of the adhesive component layer of the BEC 102. In some examples, the BEC 102 is reusable.

A communication connection is established between the BEC 102 and the computing device 104 (304). Specifically, the BEC 102 is paired with the computing device 104 utilizing the communications module 108. The communications module 108 is able to transmit data to the computing device 104 obtained from the sensor module 110. The computing device 104 receives the data such that the application 112 can appropriately process the obtained data. In some examples, the communication connection is based on a protocol utilizing by the communication module 108 (e.g., Bluetooth, Bluetooth low energy, wireless—WLAN or Wi-Fi, or radio frequency).

The BEC 102 obtains data (306). Specifically, the BEC 102 obtains data associated with the user 105 and/or the environment associated with the user 105. The BEC 102 obtains such data utilizing the sensor module 110. The BEC 102 can obtain the data continuously, or at predetermined time intervals (e.g., 1 nanosecond, 1 millisecond, 1 second, etc.). In some examples, the BEC 102 obtains the data in response to instructions from the user 105 (e.g., by providing approval to obtain data through the application 112) or automatically (e.g., upon initialization of the BEC 102). In some examples, the BEC 102 obtains the data in response to a triggering event (e.g., detected motion of the user 105, detected perspiration of the user 105). In some examples, the data obtained by the sensor module 110 is based on parameters of the sensor module 110. That is, a type of the sensor module 110 and the data-collecting abilities of the sensor module 110 (e.g., what types of data the sensor module 110 is able to detect/obtain).

In some examples, the obtained data is stored by the computing device 104 (e.g., in a memory of the computing device 104). The data can be stored for a limited time (e.g., 30 days) and then subsequently removed from storage (e.g., automatically or in response to user input). In some examples, the communications module 108 can provide the obtained data (from the sensor module 110) to a third party. For example, the communications module 108 can provide the obtained data to another computing device (e.g., associated with another user—a friend of the user 105). For example, the communications module 108 can provide the obtained data to a back-end server-computing device for appropriate processing of the data (e.g., further processing of the data above the processing performed by the computing device 104).

In some examples, the BEC 102 can be removed from the skin of the user 105 by applying water to the BEC 102 such that the components of the BEC 102 are dissolved and/or the adhesion between the BEC 102 and the skin of the user 105 is removed. In some examples, the BEC 102 can be removed from the skin of the user 105 by applying a peeling force to the BEC 102 to separate the BEC 102 from the skin of the user 105.

In an example of use of the BEC 102 (e.g., real-world implementation of the BEC 102), the user 105 adheres the BEC 102 to the skin thereof, and establishes a connection between the BEC 102 and the computing device 104. The BEC 102 is able to obtain temperature data of the user 105, and provides this temperature data to the application 112. The application 112 is able to monitor the temperature data, and when the temperature data rises above a threshold, an alert is provided to the user 105 (e.g., a notification on the computing device 104).

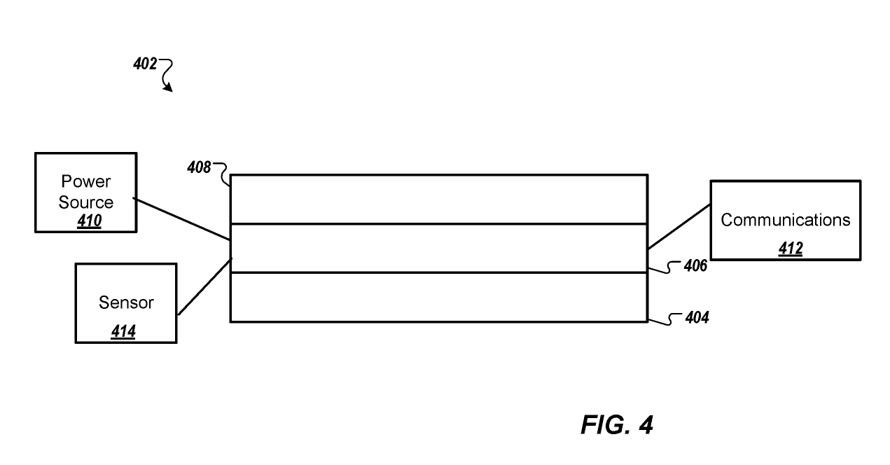

FIG. 4 is side view of a BEC 402 in physical form. Specifically, the BEC 402 (e.g., similar to the BEC 102) includes three layers: a conductive layer 404, an adhesive component layer 406, and an aesthetic layer 408 (similar to that mentioned above with respect to FIG. 2). In some examples, the adhesive component layer 406 is positioned between the conductive layer 404 and the aesthetic layer 408. The BEC 402 further includes a power source component 410, a communications module 412, and a sensor module 414 (similar to the power source component 106, the communications module 108, and the sensor module 110). The power source component 410, the communications module 412, and the sensor module 414 are coupled to the conductive layer 404, as described above.

Implementations of the present disclosure and all of the functional operations provided herein can be realized in digital electronic circuitry, or in computer software, firmware, or hardware, including the structures disclosed in this specification and their structural equivalents, or in combinations of one or more of them. Implementations of the present disclosure can be realized as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this disclosure can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. Elements of a computer can include a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the present disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

While this disclosure includes some specifics, these should not be construed as limitations on the scope of the disclosure or of what may be claimed, but rather as descriptions of features of example implementations of the disclosure. Certain features that are described in this disclosure in the context of separate implementations can also be provided in combination in a single implementation. Conversely, various features that are described in the context of a single implementation can also be provided in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the present disclosure have been described. Other implementations are within the scope of the following claims. For example, the actions recited in the claims can be performed in a different order and still achieve desirable results. A number of implementations have been described. Nevertheless, it will be understood that various modifications may be made without departing from the spirit and scope of the disclosure. For example, various forms of the flows shown above may be used, with steps re-ordered, added, or removed. Accordingly, other implementations are within the scope of the following claims.

1. A biowearable embedded circuit, comprising:
a first conductive layer that transmits electrical signals between a sensor module and one or more components of the biowearable embedded circuit;
a second adhesive component layer adhering the biowearable embedded circuit to a skin of a user; and
a third insulation layer to insulate the biowearable embedded circuit,
wherein the sensor module is coupled to the first conductive layer to obtain atmospheric, motion, communication, and/or biometric data associated with the user.

2. The circuit of claim 1, further comprising a power source coupled to the first conductive layer.

3. The circuit of claim 1, further comprising a communications module coupled to the first conductive layer.

4. The circuit of claim 1, wherein the adhesive component layer comprises a water-based adhesive.

5. The circuit of claim 1, wherein the second adhesive component layer is positioned between the first conductive layer and the third insulation layer.

6. The circuit of claim 1, further comprising an additional layer configured to minimize amperage loss of the circuit, the first conductive layer positioned between the additional layer and the second adhesive component layer.

7. The circuit of claim 1, wherein the third insulation layer is polyethylene terephthalate based composition.

8. A method of manufacturing a biowearable embedded circuit, comprising:
generating a graphics file of the biowearable embedded circuit, the graphics file including a conductive layer, an adhesive layer, and an insulation layer of the biowearable embedded circuit;
printing the graphics file to produce each layer of the biowearable embedded circuit in a specified order; and
attaching one or more components to the printed biowearable embedded circuit.

9. The method of claim 8, wherein printing the graphics file further includes printing the graphics file on a sheet including multiple biowearable embedded circuits that includes the biowearable embedded circuit, the method further comprising:
separating the biowearable embedded circuit from remaining biowearable embedded circuits of the multiple biowearable embedded circuits.

10. The method of claim 8, wherein printing the graphics file further includes:
printing the conductive layer on a base layer;
after printing the conductive layer on the base layer, printing the adhesive layer on the conductive layer; and
after printing the adhesive layer on the conductive layer, printing the insulation layer on the conductive layer.

11. The method of claim 10, wherein printing the conductive layer further includes printing the conductive layer that includes an initial state, the initial state including a permeable state.

12. The method of claim 11, wherein after attaching the one or more components to the printed biowearable embedded circuit, the conductive layer includes a second state, the second state including a non-permeable state.
